(19) United States
(12) Patent Application Publication
(10) Pub. No.: US 2024/0234814 A1
(43) Pub. Date: Jul. 11, 2024

(54) COMPOSITION

(71) Applicant: Mexichem Fluor S.A. de C.V., San Luis Potosi (MX)

(72) Inventors: Andrew Sharratt, Runcorn (GB); Ira Saxena, Runcorn (GB)

(21) Appl. No.: 18/555,765

(22) PCT Filed: Apr. 21, 2022
(86) PCT No.: PCT/EP2022/060526

§ 371 Date: Oct. 17, 2023

(30) Foreign Application Priority Data
Apr. 22, 2021 (GB) 2105744.3

(51) Int. Cl. H01M 10/0567; H01M 10/052; H01M 10/0568; H01M 10/0569

(52) U.S. Cl. CPC H01M 10/0567; H01M 10/052; H01M 10/0568; H01M 10/0569; H01M 2300/0025

(57) ABSTRACT

Use of a compound of Formula 1 in a non-aqueous battery electrolyte formulation: wherein: R is an optionally fluorinated alky group, conveniently C₁₋₆; each Y is independently H or F. X is H; a halogen, conveniently F; or an alkyl or a fluoroalkyl, conveniently C₁₋₆; each Z is independently a halogen, conveniently F; or H.

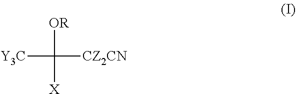

The present disclosure relates to non-aqueous electrolytic solutions for energy storage devices including batteries and capacitors, especially for secondary batteries and devices known as super capacitors.

There are two main types of battery; primary and secondary. Primary batteries are also known as non-rechargeable batteries. Secondary batteries are also known as rechargeable batteries. A well-known type of rechargeable battery is the lithium-ion battery. Lithium-ion batteries have a high energy density, no memory effect and low self-discharge.

Lithium-ion batteries are commonly used for portable electronics and electric vehicles. In the batteries, lithium ions move from the negative electrode to the positive electrode during discharge and back when charging.

Typically, the electrolytic solutions include a non-aqueous solvent and an electrolyte salt, plus additives. The electrolytic solution is typically a mixture of organic carbonates such as ethylene carbonate, propylene carbonate, fluoroethylene carbonate and dialkyl carbonates containing a lithium ion electrolyte salt. Many lithium salts with non-coordinating anions can be used as the electrolyte salt, common examples include lithium hexafluorophosphate (LiPF₆), lithium bis (fluorosulfonyl) imide “LiFSI” and lithium bis (trifluoromethanesulfonyl) imide (LiTFSI).

The electrolytic solution has to perform a number of separate roles within the battery.

The principal role of the electrolyte is to facilitate the flow of charge carriers between the cathode and anode. This occurs by transportation of metal ions within the battery from and or to one or both of the anode and cathode, where by chemical reduction or oxidation, electrical charge is liberated/adopted. Thus, the electrolytic solution needs to provide a medium which is capable of solvating and/or supporting the metal ions.

Due to the use of lithium electrolyte salts and the interchange of lithium ions with lithium metal, which is very reactive with water, as well as the sensitivity of other battery components to water, the electrolyte solution is usually non-aqueous.

Additionally, the electrolyte solvent has to have suitable rheological properties to permit/enhance the flow of ions therein, at the typical operating temperature to which a battery is exposed and expected to perform.

Moreover, the electrolyte solvent has to be as chemically inert as possible or at least react in such a way to form a stable interface on electrochemically active surfaces to help preserve the battery performance over time. In practice, however, adverse side reactions among electrolyte components and between the electrolyte and the active materials occur reducing the battery life. Often such adverse side reactions result in gas formation, which can exacerbate cell performance degradation. Therefore, every effort must be made to reduce gas generation during normal cell operation. Also, of importance within the consideration of chemical stability is flammability. Unfortunately, typical electrolyte solvents can be a safety hazard, since they often comprise a flammable material.

This can be problematic as in operation when discharging or being discharged, batteries may accumulate heat. This is especially true for high density batteries such as lithium-ion batteries and batteries with metallic lithium anodes. It is therefore desirable that the electrolyte solvent displays a low flammability, with other related properties such as a high flash point.

It is an object of the present invention to provide a non-aqueous electrolytic solution, which provides improved properties over non-aqueous electrolytic solutions of the prior art.

It is known to react and ring-open an epoxide with a fluorinated side chain by a source of cyanide, such as acetone cyanohydrin, to produce a fluorinated cyanohydrin. This is represented below:

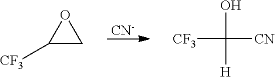

We have found that such cyanohydrins can be combined with an alkylating agent to provide a fluorinated cyanoether. Such fluorinated cyanoethers can be particularly useful as non-aqueous solvents in lithium-ion batteries.

The listing or discussion of an apparently prior-published document in this specification should not necessarily be taken as an acknowledgement that the document is part of the state of the art or is common general knowledge.

### Use Aspects

According to a first aspect of the invention there is provided the use of a compound of Formula 1 in a non-aqueous battery electrolyte formulation.

According to a second aspect of the invention there is provided the use of a non-aqueous battery electrolyte formulation comprising a compound of Formula 1 in a battery.

### Composition/Device Aspects

According to a third aspect of the invention there is provided a battery electrolyte formulation comprising a compound of Formula 1.

According to a fourth aspect of the invention there is provided a formulation comprising a metal ion and a compound of Formula 1, optionally in combination with a solvent.

According to a fifth aspect of the invention there is provided a battery comprising a battery electrolyte formulation comprising a compound of Formula 1.

### Method Aspects

According to a sixth aspect of the invention there is provided a method of increasing the flash point of a battery and/or a battery electrolyte formulation, comprising the addition of a formulation comprising a compound of Formula 1.

According to a seventh aspect of the invention there is provided a method of powering an article comprising the use of a battery comprising a battery electrolyte formulation comprising a compound of Formula 1.

According to an eighth aspect of the invention there is provided a method of retrofitting a battery electrolyte formulation comprising either (a) at least partial replacement of the battery electrolyte with a battery electrolyte formulation comprising a compound of Formula 1, and/or (b) supplementation of the battery electrolyte, with a battery electrolyte formulation comprising a compound of Formula 1.

According to a ninth aspect of the invention there is provided a method of preparing a battery electrolyte formulation comprising mixing a compound of Formula 1 with a lithium containing salt and other solvents or co-solvents.

According to a tenth aspect of the invention there is provided a method of preparing a battery electrolyte formulation comprising mixing a composition comprising a compound of Formula 1 with a lithium-containing compound.

According to an eleventh aspect of the invention there is provided a method of improving battery capacity, and/or charge transfer within a battery, and/or battery life. by the use of a compound of Formula 1.

According to a twelfth aspect of the invention there is provided a method of preparing a cyanoether, conveniently a cyanoether of Formula 1, by ring opening of an epoxide with a source of cyanide and alkylating the cyanohydrin so formed with a suitable alkylating agent to produce a cyanoether.

According to a thirteenth aspect of the invention, there is provided a method of reducing gas generation during operation of a lithium ion containing battery/cell comprising the addition of a formulation comprising a compound of Formula 1.

### Compound of Formula 1

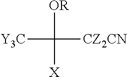

In an embodiment, R is an optionally fluorinated alky group, conveniently C₁₋₆.

In a further embodiment, each Y is independently H or F.

In an embodiment, X is H; a halogen, typically but not necessarily F; an alkyl or a fluoroalkyl; such alkyls or fluoroalkyls may typically be C₁₋₆;

In an embodiment, each Z is independently a halogen, typically but not necessarily F; or H.

In a particularly preferred embodiment, all Ys are F.

In a particularly preferred embodiment, R is CH₃, CF₃ or CH₂CF₃.

In a particularly preferred embodiment, X is H or CF₃.

In a particularly preferred embodiment, Z is H or F.

In a particularly preferred embodiment, all Ys are F; R is CH₃, CF₃ or CH₂CF₃; X is H or CF₃; and Z is H or F.

In a further preferred embodiment, where Z is a halogen it is preferably F.

### Advantages

In the aspects of the invention the electrolyte formulation has been found to be surprisingly advantageous.

The advantages of using the fluorinated cyanoether compounds of Formula 1 in electrolyte solvent compositions manifest themselves in a number of ways. Their presence can reduce the flammability of the electrolyte composition (such as when for example measured by flashpoint). Their oxidative stability makes them useful for batteries required to work in harsh conditions, they are compatible with common electrode chemistries and can even enhance the performance of these electrodes through their interactions with them. Such fluorinated cyanoether compounds may also have reduced toxicity compared to other compounds used as electrolyte solvents.

Additionally, electrolyte compositions comprising compounds of Formula 1 may have superior physical properties, including low density, low viscosity and a low melting point, yet a high boiling point with the associated advantage of little or no gas generation in use. The electrolyte formulation may wet and spread extremely well over surfaces, particularly fluorine containing surfaces and electrode surfaces; this is postulated to result from a beneficial relationship between its adhesive and cohesive forces, to yield a low contact angle.

Furthermore, electrolyte compositions that comprise compounds of Formula 1 may have superior electro-chemical properties. These include improved capacity retention, improved cyclability and capacity, improved compatibility with other battery components, e.g. separators and current collectors and with all types of cathode and anode chemistries, including systems that operate across a range of voltages, especially high voltages, and which include additives such as silicon. In addition, the electrode formulations display good solvation of metal (e.g. lithium) salts and interaction with any other electrolyte solvents present.

In a further envisaged embodiment, the invention may comprise a compound according to Formula 1. It may also comprise methods for preparing compounds according to Formula 1.

Preferred features relating to the aspects of the invention follows below.

### Metal Salts

The non-aqueous electrolytic solution further comprises a metal electrolyte salt, present in an amount of 0.1 to 99 wt % or more relative to the total mass of the non-aqueous electrolyte formulation

The metal salt generally comprises a salt of lithium, sodium, magnesium, calcium, lead, zinc or nickel.

Preferably the metal salt comprises a salt of lithium, such as those selected from the group comprising lithium hexafluorophosphate (LiPF₆), lithium perchlorate (LiClO₄), lithium tetrafluoroborate (LiBF₄), lithium triflate (LiSO₃CF₃), lithium bis (fluorosulfonyl) imide (Li(FSO₂)₂N) and lithium bis (trifluoromethanesulfonyl) imide (Li(CF₃SO₂)₂N).

Preferably, the metal salt comprises lithium hexafluorophosphate (LiPF₆), lithium bis (fluorosulfonyl) imide (Li(FSO₂)₂N) and lithium bis (trifluoromethanesulfonyl) imide (Li(CF₃SO₂)₂N). Thus, in a most preferred variant of the fourth aspect of the invention there is provided a formulation comprising lithium hexafluorophosphate (LiPF₆), lithium bis (fluorosulfonyl) imide (Li(FSO₂)₂N) and lithium bis (trifluoromethanesulfonyl) imide (Li(CF₃SO₂)₂N) and a compound of Formula 1, optionally in combination with a co-solvent.

### Other Solvents

The non-aqueous electrolytic solution may comprise an additional solvent. Preferred examples of solvents include fluoroethylene carbonate (FEC), a cyclic fluoroalkyl substituted carbonate ester, an acyclic fluoroalkyl ester, propylene carbonate (PC), dimethyl carbonate (DMC), ethylmethyl carbonate (EMC), ethylene carbonate (EC), dimethyl carbonate (DEC), vinyl carbonate (VC), cyclic polyethers such dioxolanes for example dioxolane (DOL) and analogues of containing fluorinated substituents, polyethers such as dimethoxyethane (DME), acyclic fluorinated ethers such as 1,1,2,2-tetrafluoroethoxy-1,1,2,2-tetrafluoropropane (TTE), unsaturated ethers such as trifluoropropenyl ethers or sulphur-containing compounds such as sulpholane (TMS).

Where present, the additional solvent can make up from 0.1 wt % to 99.9 wt % of the liquid component of the electrolyte.

### Additives

The non-aqueous electrolytic solution may include an additive.

Suitable additives may serve as surface film-forming agents, which form an ion-permeable film on the surface of the positive electrode or the negative electrode. This can pre-empt a decomposition reaction of the non-aqueous electrolytic solution and the electrolyte salt occurring on the surface of the electrodes, thereby preventing the decomposition reaction of the non-aqueous electrolytic solution on the surface of the electrodes.

Examples of film-forming agent additives include vinylene carbonate (VC), ethylene sulfite (ES), lithium bis (oxalato) borate (LiBOB), cyclohexylbenzene (CHB) and ortho-terphenyl (OTP). The additives may be used singly, or two or more may be used in combination.

When present the additive is present in an amount of 0.1 to 3 wt % relative to the total mass of the non-aqueous electrolyte formulation.

### Battery

The battery may comprise a primary (non-rechargeable) or a secondary (rechargeable) battery. Most preferably the battery comprises a secondary battery.

A battery comprising the non-aqueous electrolytic solutions will generally comprise several elements. Elements making up the preferred non-aqueous electrolyte secondary battery cell are described below. It is appreciated that other battery elements may be present (such as a temperature sensor); the list of battery components below is not intended to be exhaustive.

### Electrodes

The battery generally comprises a positive and negative electrode. Usually the electrodes are porous and permit metal ions (lithium ions) to move in and out of their structures by a process called insertion (intercalation) or extraction (deintercalation) or conversion (chemical reaction between metal ions and host active materials).

### Positive Electrode (Cathode)

For rechargeable batteries (secondary batteries), the term cathode designates the electrode where reduction is taking place during the discharge cycle. The cathode is also alternatively referred to as the positive electrode because it is at a higher potential (relative to a reference electrode) compared to the anode (or negative electrode). The positive electrode is generally composed of a positive electrode current collector such as a metal foil, optionally with a positive electrode active material layer disposed on the positive electrode current collector.

The positive electrode current collector may be a foil of a metal that is stable at a range of potentials applied to the positive electrode, or a film having a skin layer of a metal that is stable at a range of potentials applied to the positive electrode. Aluminium is desirable as a metal that is stable at a range of potentials applied to the positive electrode.

The positive electrode active material layer generally includes a positive electrode active material and other components such as a conductive agent and a binder. This is generally obtained by mixing the components in a solvent, applying the mixture onto the positive electrode current collector, followed by drying and rolling.

The positive electrode active material may be a lithium or a lithium-containing transition metal oxide, or it could also comprise sulphur. The transition metal element may be at least one selected from the group consisting of scandium, manganese, iron, cobalt, nickel, copper and yttrium. Of these transition metal elements, manganese, cobalt and nickel are the most preferred.

Further, in certain embodiments transition metal fluorides may be preferred.

Some of the transition metal atoms in the transition metal oxide may be replaced by atoms of a non-transition metal element. The non-transition element may be selected from the group consisting of magnesium, aluminium, lead, antimony and boron. Of these non-transition metal elements, magnesium and aluminium are the most preferred.

Preferred examples of positive electrode active materials include sulphur and lithium-containing transition metal oxides such as LiCoO₂, LiNiO₂, LiMn₂O₄, LiMnO₂, LiNi_{1-y}Co_{y}O₂ (0<y<1), LiNi_{1-y-z}Co_{y}Mn₂O₂ (0<y+z<1) and LiNi_{1-y-z}Co_{y}Al_{z}O₂ (0<y+z<1). LiNi_{1-y-z}Co_{y}Mn₂O₂ (0<y+z<0.5) and LiNi_{1-y-z}Co_{y}Al_{z}O₂ (0<y+z<0.5) containing nickel in a proportion of not less than 50 mol % relative to all the transition metals are desirable from the perspective of cost and specific capacity, or it could also comprise sulphur. These positive electrode active materials contain a large amount of alkali components and thus accelerate the decomposition of non-aqueous electrolytic solutions to cause a decrease in durability. However, the non-aqueous electrolytic solution of the present disclosure is resistant to decomposition even when used in combination with these positive electrode active materials.

The positive electrode active material may be a lithium-containing transition metal fluoride. The transition metal element may be at least one selected from the group consisting of scandium, manganese, iron, cobalt, nickel, copper and yttrium. Of these transition metal elements, manganese, cobalt and nickel are the most preferred.

Where the positive electrode comprises sulphur the electroactive material may be coated onto a suitable substrate or contained within a porous medium, such as carbon or a carbon-based matrix.

A conductive agent may be used to increase the electron conductivity of the positive electrode active material layer. Preferred examples of the conductive agents include conductive carbon materials, metal powders and organic materials. Specific examples include carbon materials such as acetylene black, Ketjen Black and graphite, metal powders such as aluminium powder, and organic materials such as phenylene derivatives.

A binder may be used to ensure good contact between the positive electrode active material and the conductive agent, to increase the adhesion of the components such as the positive electrode active material with respect to the surface of the positive electrode current collector. Preferred examples of the binders include fluoropolymers and rubber polymers, such as polytetrafluoroethylene (PTFE), polyvinylidene fluoride (PVdF) ethylene-propylene-isoprene copolymer and ethylene-propylene-butadiene copolymer. The binder may be used in combination with a thickener such as carboxymethylcellulose (CMC) or polyethylene oxide (PEO).

### Negative Electrode (Anode)

The negative electrode is generally composed of a negative electrode current collector such as a metal foil, optionally with a negative electrode active material layer disposed on the negative electrode current collector.

The negative electrode current collector may be a foil of a metal. Copper (lithium free) is suitable as the metal. Copper is easily processed at low cost, and has good electron conductivity. Depending on the active material used (e.g., with lithium titanium oxide), aluminium may also be used as the current collector.

The negative electrode may comprise carbon, such as graphite or graphene, or mixtures of carbon with other elements that can intercalate lithium, such as silicon or lithium metal.

Silicon based materials can also be used for the negative electrode either as pure silicon, or as composites with graphite. Silicon may be present in the form of nano-wires, nano-rods, particles, or flakes.

The negative electrode may include an active material layer. Where present the active material layer includes a negative electrode active material and other components such as a binder. This is generally obtained by mixing the components in a solvent, applying the mixture onto the positive electrode current collector, followed by drying and rolling.

Negative electrode active materials are not particularly limited, provided the materials can store and release lithium ions. Examples of suitable negative electrode active materials include carbon materials, metals, alloys, metal oxides, metal nitrides, and lithium-intercalated carbon and silicon. Examples of carbon materials include natural/artificial graphite, and pitch-based carbon fibres. Preferred examples of metals include lithium, silicon, tin, germanium, indium, gallium, titanium, lithium alloys, silicon alloys and tin alloys. An example of a lithium-based material includes lithium titanate (Li₂TiO₃).

As with the positive electrode, the binder may be a fluoropolymer or a rubber polymer and is desirably a rubbery polymer, such as styrene-butadiene copolymer (SBR). The binder may be used in combination with a thickener.

### Separator

A separator is preferably present between the positive electrode and the negative electrode. The separator has insulating properties. The separator may comprise a porous film having ion permeability. Examples of porous films include microporous thin films, woven fabrics and nonwoven fabrics. Suitable materials for the separators are polyolefins, such as polyethylene and polypropylene.

### Case

The battery components are preferably disposed within a protective case.

The case may comprise any suitable material which is resilient to provide support to the battery and an electrical contact to the device being powered.

In one embodiment the case comprises a metal material, preferably in sheet form, moulded into a battery shape. The metal material preferably comprises a number of portions adaptable be fitted together (e.g. by push-fitting) in the assembly of the battery. Preferably the case comprises an iron/nickel/steel-based material.

In another embodiment the case comprises a plastics material, moulded into a battery shape. The plastics material preferably comprises a number of portions adaptable be joined together (e.g. by push-fitting/adhesion) in the assembly of the battery.

Preferably the case comprises a polymer such as polystyrene, polyethylene, polyvinyl chloride, polyvinylidene chloride, or polymonochlorofluoroethylene. The case may also comprise other additives for the plastics material, such as fillers or plasticisers. In this embodiment wherein the case for the battery predominantly comprises a plastics material, a portion of the casing may additionally comprise a conductive/metallic material to establish electrical contact with the device being powered by the battery.

### Arrangement

The positive electrode and negative electrode may be wound or stacked together through a separator. Together with the non-aqueous electrolytic solution they are accommodated in the exterior case. The positive and negative electrodes are electrically connected to the exterior case in separate portions thereof.

### Module/Pack

A number/plurality of battery cells may be made up into a battery module. In a battery module the battery cells may be organised in series and/or in parallel. Typically, these are encased in a mechanical structure.

A battery pack may be assembled by connecting multiple modules together in series or parallel. Typically, battery packs include further features such as sensors and controllers, including battery management systems and thermal management systems. The battery pack generally includes an encasing housing structure to make up the final battery pack product.

### End Uses

The battery of the invention, in the form of an individual battery/cell, module and/or pack (and the electrolyte formulations therefor) are intended to be used in one or more of a variety of end products.

Preferred examples of end products include portable electronic devices, such as GPS navigation devices, cameras, laptops, tablets and mobile phones. Other preferred examples of end products include vehicular devices (as provision of power for the propulsion system and/or for any other electrical system or devices present therein) such as electrical bikes and motorbikes as well as automotive applications (including hybrid and purely electric vehicles).

Preferences and options for a given aspect, feature or parameter of the invention should, unless the context indicates otherwise, be regarded as having been disclosed in combination with any and all preferences and options for all other aspects, features and parameters of the invention.

### Preparation of the Compounds of Formula 1

In an aspect of the invention, there is provided a method on manufacturing the compounds of Formula 1 by reacting a compound of Formula 2 below:

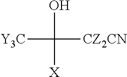

with an alkylating agent to form a compound of Formula 1:

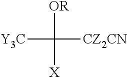

In an embodiment, R is an optionally fluorinated alky group, conveniently C₁₋₆.

In a further embodiment, each Y is independently H or F.

In an embodiment, X is H; a halogen, typically but not necessarily F; an alkyl or a fluoroalkyl; such alkyls or fluoroalkyls may typically be C₁₋₆;

In an embodiment, each Z is independently a halogen, typically but not necessarily F; or H.

In a particularly preferred embodiment, all Ys are F.

In a particularly preferred embodiment, R is CH₃, CF₃ or CH₂CF₃.

In a particularly preferred embodiment, X is H or CF₃.

In a particularly preferred embodiment, Z is H or F.

In a particularly preferred embodiment, all Ys are F; R is CH₃, CF₃ or CH₂CF₃; X is H or CF₃; and Z is H or F.

In a further preferred embodiment, where Z is a halogen it is preferably F.

In a preferred embodiment, the compound of Formula 2 is formed by ring-opening of an epoxide by a cyanide compound. A preferred example of such a cyanide compound is acetone cyanohydrin, but other sources of cyanide can be used, including metal cyanides such as potassium cyanide. In a separate or consecutive step, the compound of Formula 2 can be converted into the compound of Formula 1 using an alkylating agent; preferably the conversion of the compound of Formula 2 into the compound of Formula 1 is carried out in consecutive steps. Preferred alkylating agents include alkyl sulphates such as dimethyl sulphate, and alkyl halides such as methyl iodide.

The invention will now be illustrated with reference to the following non-limiting examples.

### EXAMPLES

General Procedure for the Ring Opening of Fluorinated Epoxides with a Source of Cyanide

Acetone cyanohydrin, triethylamine, tetrathydrofuran and epoxide were added to a 3 necked flask charge and heated at reflux with stirring for 2 hours. The progress of the reaction was monitored by ¹⁹F NMR.

Once the reaction was complete, the reaction mixture was cooled and quenched with water and then extracted twice with diethyl ether.

The ether extracts were combined and washed with 1N HCl solution and then a brine solution before being dried over anhydrous sodium sulphate. After drying, the ether was removed by distillation under vacuum.

The results are presented in Table 1 below:

| | | Acetone | Triethyl | | | | |
|---|---|---|---|---|---|---|---|
| | Epoxide | cyanohydrin | amine | THF | | Yield | Boiling |
| Example | (g) | (g) | (g) | (ml) | Product | (%) | point (° C.) |
| 1 | 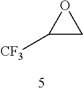 | 4.53 | 5.37 | 20 | 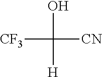 | 93 | 235-236 |
| 2 | 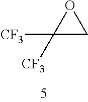 [SMILES: FC(F)(F)C1(C(F)(F)F)CO1] | 2.81 | 5.37 | 20 | 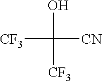 [SMILES: N#CC(O)(C(F)(F)F)C(F)(F)F] | 50.1 | 189-190 |
| 3 | 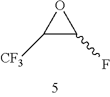 | 3.89 | 4.62 | 20 | 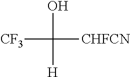 | 37.7 | — |

### General Procedure for Alkylation of a Cyanohydrin to Produce a Cyanoether

0.75 g of sodium hydroxide and 3 ml of water were added to a round bottomed flask and stirred. One this solution had cooled to room temperature, 0.03 g of tetrabutyl ammonium bromide was added and the solution was further cooled to 10° C. before 2.3 g of the cyanohydrin product of Example 1 was added dropwise whilst maintaining the temperature at 10-15° C. This solution was stirred for 30 minutes before 2.27 g of dimethyl sulphate was added dropwise whilst maintaining the temperature below 15° C. during the addition. This reaction mixture was allowed to warm to room temperature, and stirred overnight.

The reaction mixture was then extracted with 2×5 ml aliquots of diethyl ether, which were combined and dried over anhydrous Na₂SO₄ before the solvent was removed by distillation under vacuum which afforded the desired product in 71% yield:

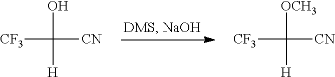 [SMILES: N#CC(O)C(F)(F)F ; COC(C#N)C(F)(F)F]

¹H NMR (400 MHz, Chloroform-d) δ 3.89 (dqd, ³J_{H-H}=7.9 Hz, ³J_{H-F}=5.9 Hz, ³J_{H-H} 4.6 Hz, 1H, CH(CF₃)(OMe)(CH₂CN)), 3.67 (s, 3H, OCH₃), 2.80-2.64 (m, 2H, CH₂CN); ¹³C NMR (101 MHz, Chloroform-d) δ 123.70 (q, ¹J_{C-F}=284.3 Hz, CF₃), 115.36 (s, CH₂CN), 75.39 (q, ²J_{C-F}=31.3 Hz, CH(CF₃)(OMe)(CH₂CN)), 61.06 (q, ⁴J_{C-F}=1.0 Hz, OCH₃), 18.94 (q, ³J_{C-F}=2.6 Hz, CH₂CN); ¹⁹F NMR (56 MHz,) δ −79.35 (d, ³J_{F-H}=6.0 Hz, CF₃).

### Example of Flash Point Measurement

The flashpoint of the cyanoether of example 1 was measured at 64° C. using the Rapid equilibrium closed cup method (ISO 3679:2015). The flashpoint for typical battery electrolyte (1M LiPF₆ in EC:EMC, 3:7, wt. %) was measured at 32° C. Therefore, addition of the cyanoether to the electrolyte will increase the flash point of the electrolyte.

### Example of Electrolyte Functionality

One of the requirements to act as an electrolyte solvent is the ability to solvate the metal ion salt, which in turn will enable the salt to dissolve in the solvent. In testing, it was found that 2.5M LiPF₆ salt could dissolve in pure cyanoether solvent of example 1. This confirms the ability for the cyanoether to be used as a battery electrolyte solvent.

### Example of Gassing Reduction

The cyanoether material synthesized in Example 1 was tested in Li-ion cells to confirm the potential for this class of molecules to reduce gas generation.

230 mAh dry Li-ion cells with artificial graphite as the anode, and NMC811 as the cathode were sourced from LiFun Technology Corporation in Hunan, China. These cells were filled with two different electrolytes: a control electrolyte without the cyanoether (control) and one with cyanoether (example electrolyte). The compositions of these electrolytes are listed below:
Control electrolyte: EC/DEC/EMC (1/1/1, % v)+1% VC+1M LiPF₆
Example electrolyte: control electrolyte+3 vol % cyanoether of example 1

Subsequently, the cells were formed using standard protocols and degassed to remove any gas generated during formation.

Following the degassing, three cells were tested for cycle life at 30° C., and 3 cells were tested at 60° C. without voltage control. As can be seen in the data below, in both cases, gas generation was reduced with the use of the cyanoether of example 1.

### Cycling at 30° C.

Cells were charge/discharge cycled at 30° C. Following the cycling test, the gas generated was measured using the Archimedes method (water displacement). As can be seen from the results in FIG. 1, there is no negative impact of the cyanoether compound on the discharge capacity during cycling. Furthermore, as seen in FIG. 2, the use of cyanoether does appear to reduce the amount of gas generated.

### Storage at 60° C.

3 cells were charged to 4.3V and stored at 60° C. for 11 days. At the end of this period, the cells were discharged (retained capacity). Charged back to 4.3V, and discharged to 2.75 V (recovered capacity). As can be seen in FIG. 3, the storage at 60 C did not have a substantive impact on the recovered capacity. However, as can be seen from the results in FIG. 4, there is a measurable decrease in the amount of gas generated.

### FIGURES

FIG. 1 shows capacity as a function of cycle number for cells filled with the control and example electrolytes as described in the text. Cycling conditions: 4.3V−2.75V, C/2 charge and C/2 discharge.

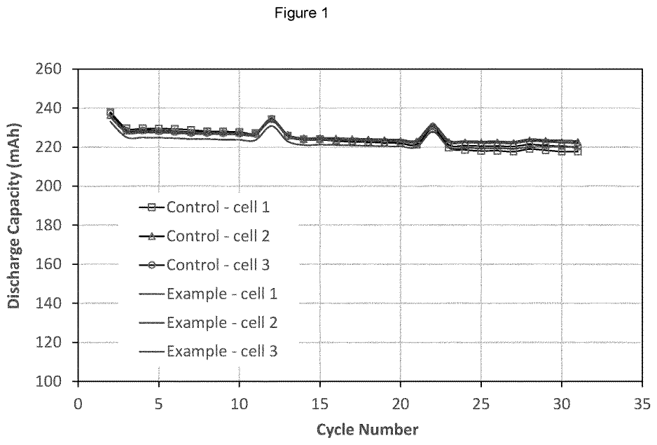

FIG. 2 shows a comparison of volume increase after cycling at 30° C. for cells with control and example electrolytes. The error bars display the range of measured values in the experiment.

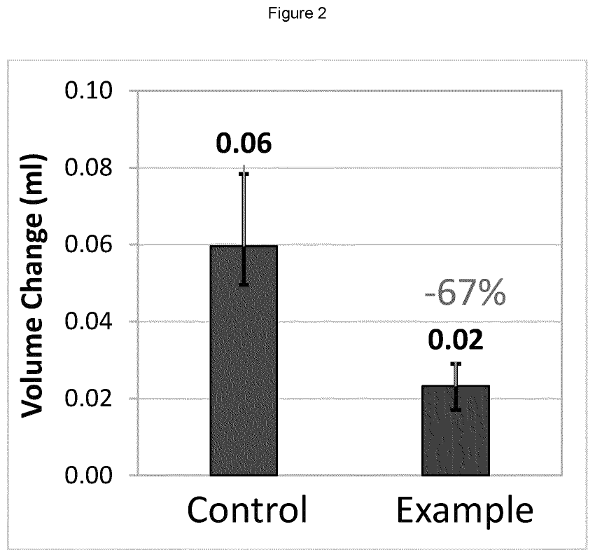

FIG. 3 shows the discharge capacity of cells with the two different electrolytes measured before storage, immediately after storage (retained capacity) and following full charge (recovered capacity). The error bars display the range of measured values in the experiment.

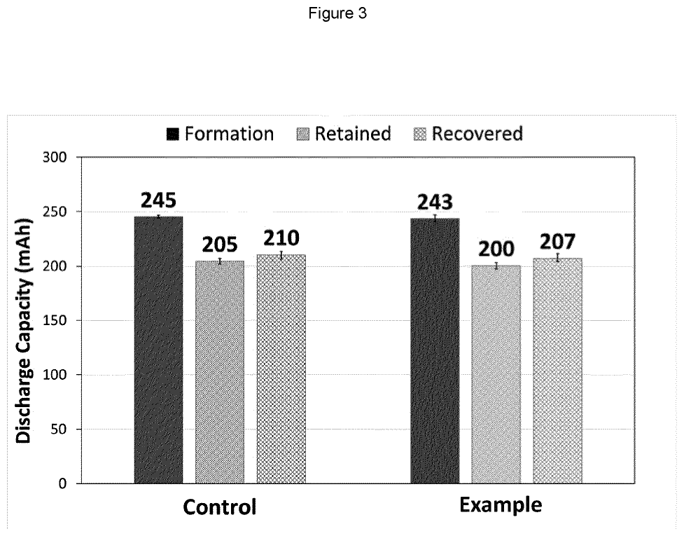

FIG. 4 shows the gas generated following storage at 60° C. (as described in FIG. 3). The error bars display the range of measured values in the experiment.

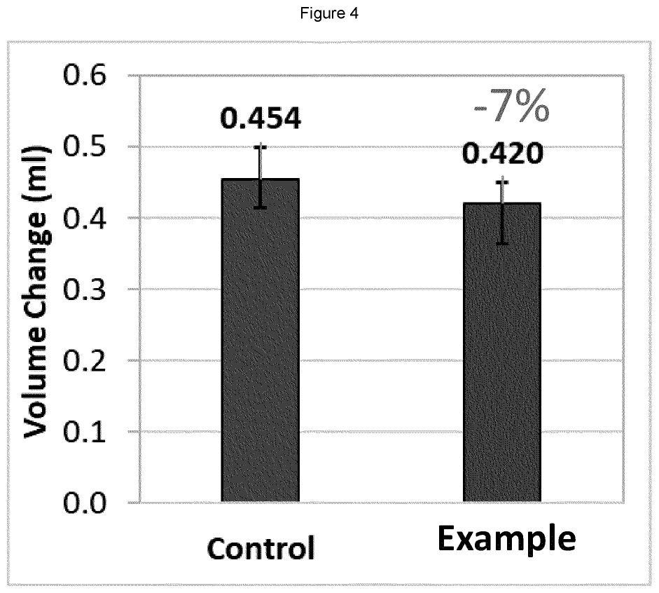

1. A method comprising providing a compound of Formula 1 in a non-aqueous battery electrolyte formulation: 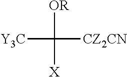
wherein: R is an optionally fluorinated alkyl group, conveniently C₁₋₆;
each Y is independently H or F.
X is H; a halogen, conveniently F; or an alkyl or a fluoroalkyl, conveniently C₁₋₆;
each Z is independently a halogen, conveniently F; or H.

2. The method of claim 1 wherein all Ys are F.

3. The method of claim 1 wherein R is CH₃, CF₃ or CH₂CF₃.

4. The method of claim 1 wherein X is H or CF₃.

5. The method of claim 1 wherein Z is H or F.

6. The method of claim 1 wherein all Ys are F; R is CH₃, CF₃ or CH₂CF₃; X is H or CF₃ and Z is H or F.

7. The method of claim 1 wherein each of the halogens in Z is F.

8. (canceled)

9. The method of claim 1 wherein the formulation comprises a metal electrolyte salt, present in an amount of 0.1 to 99 wt % or more relative to the total mass of the non-aqueous battery electrolyte formulation.

10. The method of claim 9, wherein the metal salt is a salt of lithium, sodium, magnesium, calcium, lead, zinc or nickel, or wherein the metal salt is selected from the group consisting of lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF3), lithium bis (fluorosulfonyl) imide (Li(FSO₂)₂N) and lithium bis (trifluoromethanesulfonyl) imide (Li(CF₃SO₂)₂N).

11. (canceled)

12. The method of claim 1 wherein the formulation comprises an additional solvent in an amount of from 0.1 wt % to 99.9 wt % of the liquid component of the formulation, wherein the additional solvent is selected from the group comprising fluoroethylene carbonate (FEC), a cyclic fluoroalkyl substituted carbonate ester, an acyclic fluoroalkyl ester, propylene carbonate (PC), ethylene carbonate, ethyl methyl carbonate (EMC), dimethyl carbonate (DMC), diethyl carbonate (DEC) or vinyl carbonate (VC), cyclic polyethers such dioxolanes for example dioxolane (DOL) and analogues of containing fluorinated substituents, polyethers such as dimethoxyethane (DME), acyclic fluorinated ethers such as 1,1,2,2-tetrafluoroethoxy-1,1,2,2-tetrafluoropropane (TTE), unsaturated ethers such as trifluoropropenyl ethers or sulphur containing compounds such as sulpholane (TMS).

13. (canceled)

14. A battery electrolyte formulation comprising a compound of Formula 1: 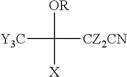
wherein: R is an optionally fluorinated alkyl group, conveniently C₁₋₆;
each Y is independently H or F.
X is H; a halogen, conveniently F; or an alkyl or a fluoroalkyl, conveniently C₁₋₆;
each Z is independently a halogen, conveniently F; or H.

15. The battery electrolyte formulation of claim 14 comprising a metal ion and a compound of Formula 1 optionally in combination with a solvent.

16. A battery comprising a battery electrolyte formulation comprising a compound of Formula 1: 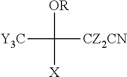
wherein: R is an optionally fluorinated alkyl group, conveniently C₁₋₆;
each Y is independently H or F.
X is H; a halogen, conveniently F; or an alkyl or a fluoroalkyl, conveniently C₁₋₆;
each Z is independently a halogen, conveniently F; or H.

17. The battery electrolyte formulation of claim 14, wherein the formulation comprises a metal electrolyte salt, present in an amount of 0.1 to 100 wt % or more relative to the total mass of the battery electrolyte formulation.

18. The battery electrolyte formulation of claim 17, wherein the metal salt is a salt of lithium, sodium, magnesium, calcium, lead, zinc or nickel, or wherein the metal salt is a salt of salt of lithium selected from the group consisting of lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF3), lithium bis (fluorosulfonyl) imide (Li(FSO₂)₂N) and lithium bis (trifluoromethanesulfonyl) imide (Li(CF₃SO₂)₂N).

19. (canceled)

20. The battery electrolyte formulation of claim 14, wherein the formulation comprises an additional solvent in an amount of from 0.1 wt % to 99.9 wt % of the liquid component of the formulation, wherein the additional solvent is selected from the group comprising fluoroethylene carbonate (FEC), a cyclic fluoroalkyl substituted carbonate ester, an acyclic fluoroalkyl ester, propylene carbonate (PC), ethylene carbonate, ethyl methyl carbonate (EMC), dimethyl carbonate (DMC), diethyl carbonate (DEC) or vinyl carbonate (VC), cyclic polyethers such dioxolanes for example dioxolane (DOL) and analogues of containing fluorinated substituents, polyethers such as dimethoxyethane (DME), acyclic fluorinated ethers such as 1,1,2,2-tetrafluoroethoxy-1,1,2,2-tetrafluoropropane (TTE), unsaturated ethers such as trifluoropropenyl ethers or sulphur containing compounds such as sulpholane (TMS).

21. (canceled)

22. A method of reducing the flammability of a battery and/or a battery electrolyte comprising the addition of a formulation comprising a compound of Formula 1: 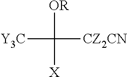
wherein: R is an optionally fluorinated alkyl group, conveniently C₁₋₆;
each Y is independently H or F.
X is H; a halogen, conveniently F; or an alkyl or a fluoroalkyl, conveniently C₁₋₆;
each Z is independently a halogen, conveniently F; or H.

23. A method of powering an article comprising electrically coupling the article with a battery comprising a battery electrolyte formulation comprising a compound of Formula 1: 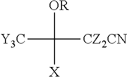
wherein: R is an optionally fluorinated alkyl group, conveniently C₁₋₆;
each Y is independently H or F.
X is H; a halogen, conveniently F; or an alkyl or a fluoroalkyl, conveniently C₁₋₆;
each Z is independently a halogen, conveniently F; or H.

24. A method of retrofitting a battery electrolyte formulation comprising either (a) at least partial replacement of the battery electrolyte with a battery electrolyte formulation comprising a compound of Formula 1, and/or (b) supplementation of the battery electrolyte with a battery electrolyte formulation comprising a compound of Formula 1: 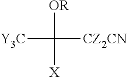
wherein: R is an optionally fluorinated alkyl group, conveniently C₁₋₆;
each Y is independently H or F.
X is H; a halogen, conveniently F; or an alkyl or a fluoroalkyl, conveniently C₁₋₆;
each Z is independently a halogen, conveniently F; or H.

25. A method of preparing a compound of Formula 1 in which a compound of Formula 2: 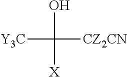
is treated with an alkylating agent, wherein:
each Y is independently H or F.
X is H; a halogen, conveniently F; or an alkyl or a fluoroalkyl, conveniently C₁₋₆;
each Z is independently a halogen, conveniently F; or H.

26. A method of preparing a battery electrolyte formulation comprising mixing an electrolyte with the compound of Formula 1 according to claim 1.

27. A method of improving battery capacity, and/or charge transfer within a battery, and/or battery life, the method comprising introducing into a battery a formulation comprising the compound of Formula 1 according to claim 1.

28. The method according to claim 22, wherein the formulation comprises a metal electrolyte salt, present in an amount of 0.1 to 20 wt % or more relative to the total mass of the battery electrolyte formulation.

29. The method according to claim 28, wherein the metal salt is a salt of lithium, sodium, magnesium, calcium, lead, zinc or nickel, or wherein the metal salt is a salt of salt of lithium selected from the group comprising lithium hexafluorophosphate (LiPF₆), lithium hexafluoroarsenate monohydrate (LiAsF₆), lithium perchlorate (LiClO₄), lithium tetrafluoroborate (LiBF₄), lithium triflate (LiSO₃CF3), lithium bis (fluorosulfonyl) imide (Li(FSO₂)₂N) and lithium bis (trifluoromethanesulfonyl) imide (Li(CF₃SO₂)₂N).

30. (canceled)

31. The method according to claim 22, wherein the formulation comprises an additional solvent in an amount of from 0.1 wt % to 99.9 wt % of the liquid component of the formulation, wherein the additional solvent is selected from the group comprising fluoroethylene carbonate (FEC), propylene carbonate (PC) and ethylene carbonate (EC), cyclic polyethers such as dioxolanes for example dioxolane (DOL) and analogues of containing fluorinated substituents, polyethers such as dimethoxyethane (DME), acyclic fluorinated ethers such as 1,1,2,2-tetrafluoroethoxy-1,1,2,2-tetrafluoropropane (TTE), unsaturated ethers such as trifluoropropenyl ethers or sulphur containing compounds such as sulpholane (TMS).

32. (canceled)
